(19) United States
(12) Patent Application Publication
(10) Pub. No.: US 2024/0104523 A1
(43) Pub. Date: Mar. 28, 2024

(54) VARIABLE TOKEN GENERATION BASED ON CARBON DIOXIDE SEQUESTRATION

(71) Applicant: Carbon Accounting Co, LLC, Casper, WY (US)

(72) Inventors: Ari Bernstein, New York, NY (US); Jeremy Robert Friesen, Cheyenne, WY (US); Terrence Manning, Casper, WY (US)

(21) Appl. No.: 18/371,803

(22) Filed: Sep. 22, 2023

Related U.S. Application Data
(60) Provisional application No. 63/408,986, filed on Sep. 22, 2022.

(51) Int. Cl. G06Q 20/06; G01N 33/00; G06Q 20/38; G06Q 30/0645

(52) U.S. Cl. CPC G06Q 20/065; G01N 33/004; G06Q 20/3829; G06Q 30/0645

(57) ABSTRACT

A system configured to variably generate tokens in response to the sequestration of carbon dioxide and a corresponding method are disclosed herein.

### BACKGROUND

### 1. Field

The present disclosure relates to the removal of carbon dioxide from air and sequestration of the extracted carbon dioxide.

### 2. Description of Related Prior Art

U.S. Pat. No. 11,389,761 discloses a System and Method for improving the performance and lowering the cost of atmospheric carbon dioxide removal by direct air capture. The system and method includes a plurality of carbon capture containers, a plurality of fans, an air diverter, and a velocity stack. Each of the carbon capture containers has an outwardly facing side and an inwardly facing side with the inwardly facing side facing an enclosed space. The fans are disposed adjacent to the carbon capture containers. The fans are arranged to move air through the carbon capture containers in a first direction from the outwardly facing side into the enclosed space. The air diverter is disposed within the enclosed space and receives the air flowing in the first direction and redirects the air to flow in a second direction that is angled upwardly from the first direction. The velocity stack is disposed on top of the enclosed space and is configured to accelerate the flow of the air in the second direction.

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

### SUMMARY

In summary, a system configured to variably generate tokens in response to the sequestration of carbon dioxide and a corresponding method are disclosed herein.

### BRIEF DESCRIPTION OF THE DRAWINGS

The detailed description set forth below references the following drawings:

FIG. 1 is a schematic representation of a system according to an exemplary embodiment of the present disclosure;

FIG. 2 is simplified flow diagram of a method according to an exemplary embodiment of the present disclosure; and

FIG. 3 is a schematic representation of carbon sequestration and a plurality of sensors that can be utilized for monitoring the carbon sequestration.

### DETAILED DESCRIPTION

The present disclosure, as demonstrated by the exemplary embodiment described below, can provide a system, referenced at 10, configured to generate tokens in response to the sequestration of carbon dioxide and a corresponding method. The present disclosure can provide an incentive to sequester carbon dioxide and give parties who wish to offset carbon dioxide emissions with a transparent and verifiable way to do so. The present disclosure can provide an incentive to sequester carbon dioxide by creating a digital asset (tokens on a distributed ledger) that will initially be owned by the sequesterer of carbon dioxide and can be sold to other parties that wish to offset their carbon emissions. The resulting digital asset's provenance, ownership, and chain of custody will be recorded on a distributed ledger, a computer network with multiple independent nodes. This feature makes the ledger resistant to hacking, and thus protects against theft or forgery of assets without the need for an expensive centralized agent, like a bank. This mechanism of protection against theft or forgery enhances the value of the digital asset by protecting ownership. It also enhances value by providing transparent credibility that each of the digital assets represents a verified volume of sequestered carbon dioxide.

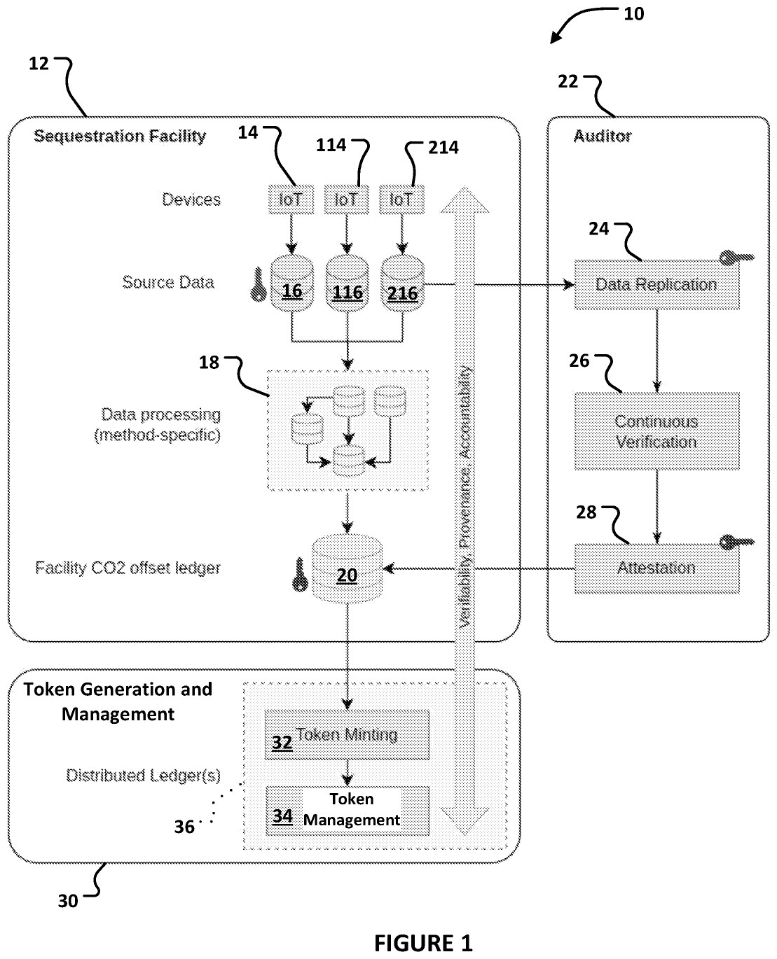

Carbon capture is the process of separating carbon dioxide from ambient air in the atmosphere or industrial effluent at its point of emission. Geological carbon sequestration is the process of storing that captured carbon dioxide underground through injection into subsurface aquifers. With reference now to FIG. 1, a carbon dioxide sequestration facility, shown schematically, is referenced at 12. Sequestered carbon dioxide can be directed into the ground. The carbon dioxide sequestration facility 12 can practice any process of carbon dioxide removal from the air. For example, the carbon dioxide sequestration facility 12 can practice solid direct air capture (“S-DAC”) using solid adsorbents operating through an adsorption/desorption cycling process. Adsorption can take place at ambient temperature and pressure. Desorption occurs through a temperature—vacuum swing process and CO₂ is released at low pressure and moderate temperature (80-100° C.). A single adsorption/desorption unit applying S-DAC typically has a capture capacity of tons of CO₂ per year. Further, a S-DAC plant can be modular and can include as many units as desired. For example, a currently operating S-DAC plant captures four thousand ton of CO₂ a year.

In another example, the carbon dioxide sequestration facility 12 can practice liquid direct air capture (“L-DAC”). L-DAC is executed through two closed chemical loops. The first loop brings atmospheric air into contact with an aqueous basic solution such as potassium hydroxide to capture the CO₂. The second loop releases the captured CO2 from the solution in a series of stages or units operating at a relatively high temperature, between 300° C. and 900° C. A large plant applying L-DAC can capture around one MtCO₂/year from the atmosphere. Weather conditions local to the plant may dictate water top-up. For instance, around 4.7 ton of water per ton of captured CO₂ would be required for an L-DAC plant operating at ambient conditions of 64% relative humidity and 20° C., by way of example and not limitation.

U.S. Pat. No. 11,389,761 discloses a method of carbon removal that could be applied in one or more embodiments of the present disclosure. Electro-swing adsorption (“ESA”) and membrane-based DAC (“m-DAC”) are also methods of carbon removal that could be applied in one or more embodiments of the present disclosure. ESA is based on an electrochemical cell in which a solid electrode adsorbs CO₂ when negatively charged and releases the CO₂ when a positive charge is applied to the electrode. This approach can separate CO₂ from highly concentrated sources of CO₂ as well as from air, requires limited space since the cells are stackable, and operates without additional equipment for conditioning or pumping, such as required for L-DAC. The ESA separation process has been tested at lab scale for CO₂ concentrations from 10% to 0.6% with an efficiency of around 90%.

Carbon capture methods also include point source capture. Point source carbon capture involves the capture of CO₂ from the emission of flue stacks, for example, where CO₂ is much more concentrated. The three main types of point source capture are pre-combustion capture, oxy-fuel combustion capture, and post-combustion capture. It is noted that the present disclosure for monitoring carbon sequestration is applicable to all processes that are practiced for capturing CO₂.

Referring now to FIG. 1, the exemplary carbon dioxide sequestration facility 12 includes devices 14, 114, 214 that can sense one or more conditions associated with the capturing of CO₂ and/or the removal of CO₂ from air. The devices 14, 114, 214 can incorporate sensors or can be data acquisition devices communicating with sensors that can sense one or more conditions associated with the capturing of CO₂ and/or the removal of CO₂ from air. It can be desirable that such sensors are tamper-proof. The devices 14, 114, 214 can connect and exchange data with other devices and systems over a network, such as the internet or another communications network, and are individually addressable.

The exemplary carbon dioxide sequestration facility 12 also includes one or more databases 16, 116, 216. The one or more databases 16, 116, 216 can receive data signals from the devices 14, 114, 214 and store the data. The one or more databases 16, 116, 216 can include readable storage media and communication media. Computer readable storage media can be non-transitory in nature, and may include volatile and non-volatile, and removable and non-removable media implemented in any method or technology for storage of information, such as computer-readable instructions, data structures, program modules or other data. Computer readable storage media may further include RAM, ROM, erasable programmable read-only memory (EPROM), electrically erasable programmable read-only memory (EEPROM), flash memory or other solid state memory technology, CD-ROM, digital versatile disks (DVD), or other optical storage, magnetic cassettes, magnetic tape, magnetic disk storage or other magnetic storage devices, or any other medium that can be used to store the desired information and which can be accessed by controller 170. Communication media may embody computer readable instructions, data structures or other program modules. By way of example, and not limitation, communication media may include wired media such as a wired network or direct-wired connection, and wireless media such as acoustic, RF, infrared and other wireless media. Combinations of any of the above may also be included within the scope of computer readable media.

The facility 12 can also include a computing device 18 configured to process and analyze the data stored in the one or more databases 16, 116, 216. The nature of the processing will be based on the nature of the sequestration process, such that an output of such processing will be the determination of the amount of carbon dioxide directed into the ground. For the purposes of the present disclosure, computing device 18 may represent practically any type of computer, computer system, controller, logic controller, or other programmable electronic device, and may in some embodiments be implemented using one or more networked computers or other electronic devices, whether located locally or remotely with respect to facility 12. The computing device 18 typically includes a central processing unit (CPU) including at least one microprocessor coupled to a memory, which may represent the random access memory (RAM) devices comprising the main storage of computing device 18, as well as any supplemental levels of memory, e.g., cache memories, non-volatile or backup memories (e.g., programmable or flash memories), read-only memories, etc. In addition, the memory may be considered to include memory storage physically located elsewhere in computing device 18, e.g., any cache memory in a processor in CPU, as well as any storage capacity used as a virtual memory, e.g., as stored on a mass storage device or on another computer or electronic device coupled to computing device 18. The computing device 18 may also include one or more mass storage devices, e.g., a floppy or other removable disk drive, a hard disk drive, a direct access storage device (DASD), an optical drive (e.g., a CD drive, a DVD drive, etc.), and/or a tape drive, among others. Furthermore, the computing device 18 may include an interface with one or more networks (e.g., a LAN, a WAN, a wireless network, and/or the Internet, among others) to permit the communication of information to the other portions and components of the facility 12 as well as with other, remote computers and electronic devices. The computing device 18 operates under the control of an operating system, kernel and/or firmware and executes or otherwise relies upon various computer software applications, components, programs, objects, modules, data structures, etc. Moreover, various applications, components, programs, objects, modules, etc. may also execute on one or more processors in another computer coupled to computing device 18, e.g., in a distributed or client-server computing environment, whereby the processing required to implement the functions of a computer program may be allocated to multiple computers over a network.

The facility 12 can also include a ledger database 20. The output(s) of processing operations of the computing device 18 can be stored in the ledger database 20. The ledger database 20 can thus store the data that is the determination of the amount of carbon dioxide directed into the ground. The descriptions above of optional, physical attributes of the one or more databases 16, 116, 216 are applicable to the ledger database 20 as well.

The exemplary system 10 also includes an auditor facility 22. The auditor facility 22 can be a physical facility or a virtual facility. The auditor facility 22 can be physically remote from the facility 12. The auditor facility 22 can include one or more computing devices in the form of one or more servers. The descriptions above of optional, physical attributes of the computing device 18 are applicable to exemplary auditor facility 22 as well.

The exemplary auditor facility 22 includes a data replication service 24. A “service,” as used herein, includes logic but is not limited to hardware, firmware, software and/or combinations of each to perform a function(s) or an action(s), and/or to cause a function or action from another component. For example, based on a desired application or need, a service applying logic may include a software controlled microprocessor, discrete logic such as an application specific integrated circuit (ASIC), a programmable/programmed logic device, memory device containing instructions, or the like, or combinational logic embodied in hardware. Logic for performing a service may also be fully embodied as software that performs the desired functionality when executed by a processor. The data replication service 24 and the one or more databases 16, 116, 216 can form a trust relationship and exchange cryptographic keys to securely exchange the data stored by the one or more databases 16, 116, 216.

The exemplary auditor facility 22 also includes a continuous verification service 26. The continuous verification service 26 can perform processing operations similar and/or identical to the processing operations performed by the computing device 18, to determine the amount of CO₂ directed into the ground. Further, the continuous verification service 26 can be configured to additionally perform processing operations that assess the quality and veracity of the data communicated by the devices 14, 114, 214.

The exemplary auditor facility 22 also includes an attestation service 28. The attestation service 28 and the ledger database 22 can form a trust relationship and exchange cryptographic keys to securely exchange the data stored by the ledger database 22. Further, in one or more embodiments of the present disclosure, the attestation service 28 and the ledger database 22 can cooperate to define a distributed ledger recording the amount of CO₂ directed into the ground. The attestation service 28 can be configured to compare data received from the ledger database 22 against the data generated by the continuous verification service 26 to confirm identity.

In one or more embodiments of the present disclosure, ledger databases of more the one sequestration facility like facility 12 and attestation services of more than one auditor facility such as auditor facility 22 can cooperate to define a distributed ledger recording the amount of CO₂ directed into the ground. For the purposes of the present disclosure, a distributed ledger can be a file that is saved on multiple computing devices, wherein any change to the ledger must be validated by all the computing devices of the ledger. This operation inhibits a bad actor from altering the information on one computing for their private benefit.

It is noted that this first distributed ledger, a “verification ledger,” however defined, will record raw data from the CO₂ extraction monitoring sensors as well as algorithms that interpret that data, and the output of those algorithms, which will be a quantity of carbon dioxide which is verified to have been sequestered. Information on this ledger will be publicly available but encrypted. The purpose for this encryption is to protect proprietary information including information about the subsurface where the carbon dioxide is stored and the algorithms used to interpret the data from the monitoring sensors. Only necessary and trusted parties, like the U.S. Environmental Protection Agency (“EPA”) and its foreign counterparts will have access to this information.

The exemplary system 10 also includes a token generation and management facility 30. The token generation and management facility 30 can be a physical facility or a virtual facility. The token generation and management facility 30 can be physically remote from the facility 12 and from the auditor facility 22. The token generation and management facility 30 can include one or more computing devices, such as in the form of one or more servers. The descriptions above of optional, physical attributes of the computing device 18 are applicable to exemplary token generation and management facility 30 as well.

The exemplary token generation and management facility 30 and the ledger database 22 can form a trust relationship and exchange cryptographic keys to securely exchange the data stored by the ledger database 22. The exemplary token generation and management facility 30 can include a token minting service 32. Based on the data contained in the ledger database 22 as well as changes thereto, which is the data that is the determination of the amount of carbon dioxide that has been sequestered, the token minting service 32 can generate tokens. Each token can correspond to an amount of carbon dioxide that has been sequestered. The correlation between a token and corresponding amount of carbon dioxide that has been sequestered can be determined or established as desired.

The exemplary token generation and management facility 30 can include a token management service 34. The exemplary token management service 34 can maintain data associated with holder and/or owner of each token generated by the token minting service 32. The exemplary and schematically-shown token generation and management facility 30 can itself include a plurality of computing devices to thus define a distributed ledger referenced by 36.

This second distributed ledger 36 can be viewed as a “coin ledger” or “token ledger.” It can create a digital token, for example, whenever the verification ledger 22 indicates that another ton (or more than one ton) of carbon dioxide has been sequestered. The data on the coin ledger can be publicly available and unencrypted. Users from the public who access the coin ledger could be able to, among other things, buy, sell, and retire coins from circulation. In one or more embodiments of the present disclosure, the number of circulating and retired coins on the coin ledger shall never exceed the number of tons of carbon dioxide that the verification ledger records as having been stored. This latter quality increases the likelihood that the coins will tend to be scarce. It will also inhibit the creation of coins without the sequestering of carbon dioxide. For example, only when carbon dioxide is verified to have been sequestered can a coin be minted.

The two, distributed ledgers will enhance transparency and credibility for all of the carbon offsets represented on the coin ledger. Even though the public may not be able to decrypt the data on the verification ledger in one or more embodiments, the information on the verification ledger will be accurate because the decryption key will be provided to trusted actors like the EPA and also because the number of tokens on the coin ledger may not ever exceed the number of tons of carbon dioxide stored. This will inhibit double-counting or falsely claimed carbon offsetting.

The present disclosure possesses advantages over the prior art in at least two ways. First, in the field of carbon offset registry, the present disclosure solves multiple problems including opacity around the accuracy of how many tons of carbon have been removed from the atmosphere; falsely claimed carbon offsets; and double counting of carbon removal. Second, in the field of digital assets, like Ethereum, the present disclosure solves the problem of potentially unlimited tokens, which reduces the value of tokens in circulation. Because tokens can only be minted when a real world activity has already taken place, and there are financial and physical limits to the rate at which the real world activity can occur, a real limit exists to the number of tokens that can be minted.

The present disclosure further comprises a method of variably minting tokens. Variably minting tokens can further incentivize the performance of the real-world activity as early and much as possible. This is encouraged by progressively requiring an ever-greater amount of the real-world activity to be performed to mint one token on the coin ledger, while raising the value of work already performed. For example, at the outset, every ton of carbon dioxide that is stored may result in the minting of one token. As more tons are stored, the system 10 can be operated such that the storage of more than one ton is required to mint one token. Further, as the tonnage requirement to mint a token is increased, the number of carbon offsets represented by existing tokens can be increased by a proportional amount.

A progressive minting schedule could apply a number of different formulas in various embodiments of the present disclosure. One possible formula could be derived from an anticipated learning curve of carbon sequestration, such as Wright's Law. Wright's Law provides a framework for forecasting cost declines as a function of cumulative production and holds that for every cumulative doubling of units produced costs will drop by a constant percentage. In one example, with every doubling of carbon dioxide that has been sequestered underground, the cost of sequestering a ton of carbon may drop by 15%. The progressive minting schedule could be structured so that, after the first hundred million tons, every time the number of sequestered tons recorded on the verification ledger is doubled, 20% more carbon dioxide must be stored to mint one token. Additionally, already minted tokens would then represent 25% more carbon offsets than they did in the previous minting epoch. The escalating value of existing tokens thus provides a positive incentive to sequester carbon dioxide as early and as much as possible. Further, this escalation would more than offset any inflationary effect of the learning curve lowering the cost of generating new carbon offsets through carbon sequestration. An example of such a progressive minting schedule is provided in the table below.

| Minting | Coins | Tons per | Tons to | Coins | Total | Total Coins |
|---|---|---|---|---|---|---|
| Epoch | per Ton | Coin | Milestone | in Epoch | Tonnes | Minted |
| 1 | 1 | 1.0000 | 100,000,000 | 100,000,000 | — | 100,000,000 |
| 2 | 0.8 | 1.2500 | 200,000,000 | 160,000,000 | 100,000,000 | 260,000,000 |
| 3 | 0.64 | 1.5625 | 400,000,000 | 256,000,000 | 300,000,000 | 516,000,000 |
| 4 | 0.512 | 1.9531 | 800,000,000 | 409,600,000 | 700,000,000 | 925,600,000 |
| 5 | 0.4096 | 2.4414 | 1,600,000,000 | 655,360,000 | 1,500,000,000 | 1,580,960,000 |
| 6 | 0.32768 | 3.0518 | 3,200,000,000 | 1,048,576,000 | 3,100,000,000 | 2,629,536,000 |
| 7 | 0.262144 | 3.8147 | 6,400,000,000 | 1,677,721,600 | 6,300,000,000 | 4,307,257,600 |
| 8 | 0.2097152 | 4.7684 | 12,800,000,000 | 2,684,354,560 | 12,700,000,000 | 6,991,612,160 |
| 9 | 0.16777216 | 5.9605 | 25,600,000,000 | 4,294,967,296 | 25,500,000,000 | 11,286,579,456 |
| 10 | 0.134217728 | 7.4506 | 51,200,000,000 | 6,871,947,674 | 51,100,000,000 | 18,158,527,130 |
| 11 | 0.107374182 | 9.3132 | 102,400,000,000 | 10,995,116,278 | 102,300,000,000 | 29,153,643,407 |
| 12 | 0.085899346 | 11.6415 | 204,800,000,000 | 17,592,186,044 | 204,700,000,000 | 46,745,829,452 |
| 13 | 0.068719477 | 14.5519 | 409,600,000,000 | 28,147,497,671 | 409,500,000,000 | 74,893,327,123 |
| 14 | 0.054975581 | 18.1899 | 819,200,000,000 | 45,035,996,274 | 819,100,000,000 | 119,929,323,397 |
| 15 | 0.043980465 | 22.7374 | 1,638,400,000,000 | 72,057,594,038 | 1,638,300,000,000 | 191,986,917,434 |
| 16 | 0.035184372 | 28.4217 | 3,276,800,000,000 | 115,292,150,461 | 3,276,700,000,000 | 307,279,067,895 |

The present disclosure thus enhances incentives for sequestering carbon dioxide by providing an inherent financial return for those who buy tokens and hold them rather than retire them to claim carbon offsets. For example, it is expected that most of the demand for tokens minted by the system will come from companies or other parties to offset their carbon emissions. To do so, these parties will retire tokens (take them out of circulation) so that these tokens can never be used for another purpose. Only by retiring tokens can parties claim to have used the tokens to offset carbon emissions. This primary market is therefore a consumptive market for the tokens. However, the consumptive market creates no incentive to buy the tokens by parties that don't need to offset carbon emissions. A progressive minting schedule bridges this gap by providing a speculative incentive to purchase tokens and hold them until the minting epoch progresses and each coin represents a greater number of carbon offsets.

For example, in the formula represented in the table above, each coin is expected to increase in the number of carbon offsets it represents by over twenty times. Assuming the market demand for carbon offsets remains constant, and the supply of carbon offsets increases, this escalation factor provides a profit incentive to buy and hold tokens. It is thus desirable for the proliferation of a secondary market in tokens that represent the completion of a real-world activity whose cost will decline with repletion.

The subject matter of the present disclosure has the advantage over prior art in that it creates a speculative incentive to buy and hold tokens representing a real-world activity like carbon sequestration. Existing art does not provide a speculative incentive for undertaking the real-world activity. The subject matter of the present disclosure provides such an incentive.

A further advantage provided by the subject matter of the present disclosure is that the difficulty of minting a token increases at an exponential rate rather than a hyperbolic rate, as applied, for example, by Bitcoin's progressive minting schedule. A hyperbolic rate can make it prohibitively expensive to mint new tokens. An exponential rate of growth does not cause this problem because the increase to infinite value does not occur on a finite time frame as it does in a hyperbolic growth curve.

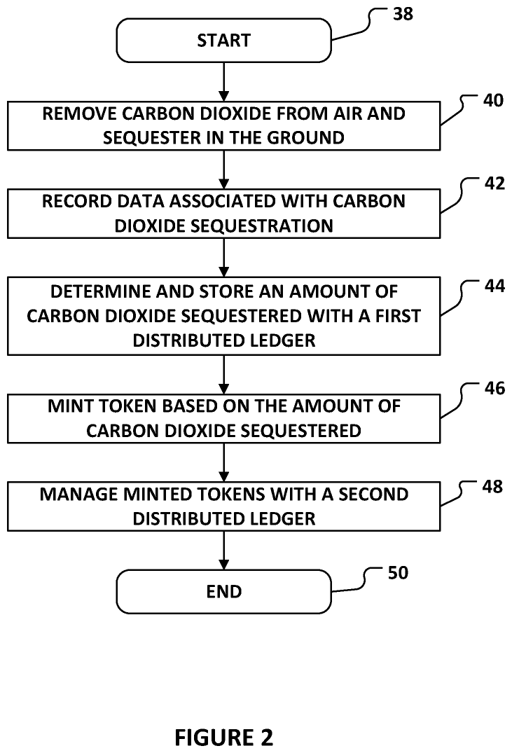

FIG. 2 is a simplified flow diagram illustrating a method that can be carried out in at least some embodiments of the present disclosure. The flow diagram displays a flowchart that illustrates the architecture, functionality, and operation of possible implementations of systems, methods, and computer program products according to various embodiments of the present disclosure. In this regard, each block in the flowchart may represent a module, segment, or portion of code, which comprises one or more executable instructions for implementing the specified logical function(s). It will also be noted that each block of the flowchart, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart or blocks.

The method starts at step 38. At step 40, carbon dioxide is removed and sequestered in the ground, such as by the exemplary facility 12. At step 42, data associated with the carbon dioxide sequestration is recorded, such as in the exemplary databases 16, 116, and 216 and the exemplary data replication service 24. At step 44, an amount of carbon dioxide sequestered is determined and stored in a first distributed ledger, such as in the exemplary ledger database 20 and the exemplary attestation service 28. At step 46, one or more tokens are minted based on the amount of carbon dioxide sequestered and also based on the number of previously minted tokens, such as by the exemplary token minting service 32. At step 48, the one or more tokens are managed, such as by the exemplary token management service 34. The exemplary process ends at step 50.

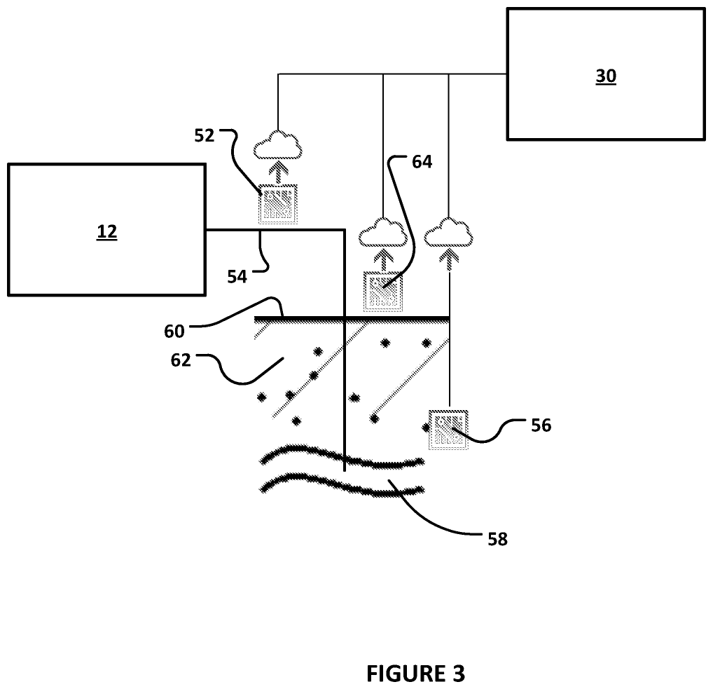

The devices 14, 114, 214 have been described above as monitoring capturing equipment. The present disclosure further provides a plurality of sensors that can be utilized for the monitoring of carbon sequestration. Referring now to FIG. 3, a first sensor or set of sensors referenced at 52 can be utilized to monitor the stream of gas coming from the capture facility 12, via a pipeline 54. The exemplary first sensor(s) 52 is configured to monitor the identity of the gas passing through the pipeline to confirm the gas is CO₂ and can also be configured to continuously monitor the volumetric flow rate of the gas passing through the pipeline. The exemplary first sensor(s) 52 is an IoT-enable sensor whereby the exemplary first sensor(s) 52 can transmit data corresponding to sensed conditions to a network and thus to the token generation and management facility 30 and or the ledger database 20 so that the first distributed ledger can be adjusted if necessary or desired.

The present disclosure further provides a second sensor or set of sensors referenced at 56 that can be utilized to monitor geological conditions around the sequestration site, referenced at 58. The sequestration site 58 is an aquifer below the surface 60 of the ground 62. The second sensor(s) 56 can be, for example, configured to monitor the concentration of CO₂ in the target formation, look for ruptures in the rock that seals the formation in which the CO₂ is injected, and track the migration of CO₂ from its injection point. The present disclosure further provides a third sensor or set of sensors referenced at 64 that can be utilized to monitor CO₂ levels at or near the surface for further verification that there is no leakage. The exemplary sensor(s) 56 and 64 are IoT-enable sensors whereby the exemplary sensor(s) 56 and 64 can transmit data corresponding to sensed conditions to a network and thus to the token generation and management facility 30 and/or the ledger database 20 so that the first distributed ledger can be adjusted if necessary or desired.

The use of any and all examples, or exemplary language (e.g., “such as”) provided herein is intended merely to be illustrative and does not pose a limitation on the scope of any invention disclosed herein unless otherwise claimed. As used herein, the singular forms “a,” “an,” and “the” include plural referents unless the context clearly dictates otherwise. Unless indicated otherwise by context, the term “or” is to be understood as an inclusive “or.” Terms such as “first”, “second”, “third”, etc. when used to describe multiple devices or elements, are so used only to convey the relative actions, positioning and/or functions of the separate devices, and do not necessitate either a specific order for such devices or elements, or any specific quantity or ranking of such devices or elements. Use of the terms “about” or “approximately” are intended to describe values above and/or below a stated value or range, as would be understood by one having ordinary skill in the art in the respective context. In some instances, this may encompass values in a range of approx. +/−10%; in other instances there may be encompassed values in a range of approx. +/−5%; in yet other instances values in a range of approx. +/−2% may be encompassed; and in yet further instances, this may encompass values in a range of approx. +/−1%.

It will be understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof, unless indicated herein or otherwise clearly contradicted by context. Recitations of a value range herein, unless indicated otherwise, serves as a shorthand for referring individually to each separate value falling within the stated range, including the endpoints of the range, each separate value within the range, and all intermediate ranges subsumed by the overall range, with each incorporated into the specification as if individually recited herein. Unless indicated otherwise, or clearly contradicted by context, methods described herein can be performed with the individual steps executed in any suitable order, including: the precise order disclosed, without any intermediate steps or with one or more further steps interposed between the disclosed steps; with the disclosed steps performed in an order other than the exact order disclosed; with one or more steps performed simultaneously; and with one or more disclosed steps omitted.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to a particular embodiment disclosed herein as the best mode contemplated for carrying out this present disclosure, but that the present disclosure will be viewed as covering any embodiment falling within the scope of the appended claims. Also, the right to claim a particular sub-feature, sub-component, or sub-element of any disclosed embodiment, singularly or in one or more sub-combinations with any other sub-feature(s), sub-component(s), or sub-element(s), is hereby unconditionally reserved by the Applicant. Also, particular sub-feature(s), sub-component(s), and sub-element(s) of one embodiment that is disclosed herein can replace particular sub-features, sub-components, and sub-elements in another embodiment disclosed herein or can supplement and be added to other embodiments unless otherwise indicated by the drawings or this specification. Further, the use of the word “can” in this document is not an assertion that the subject preceding the word is unimportant or unnecessary or “not critical” relative to anything else in this document. The word “can” is used herein in a positive and affirming sense and no other motive should be presumed. More than one “invention” may be disclosed in the present disclosure; an “invention” is defined by the content of a patent claim and not by the content of a detailed description of an embodiment of an invention.

hat is claimed is:

1. A system configured to variably generate tokens in response to the sequestration of carbon dioxide as disclosed herein.

2. A method of variably generating tokens in response to the sequestration of carbon dioxide as disclosed herein.

3. A system configured to variably generate tokens in response to the sequestration of carbon dioxide comprising:
a sequestration facility having:
one or more devices configured to can sense one or more conditions associated with the capturing and removing of CO₂ from air and to emit data corresponding to sensed conditions,
one or more databases configured to receive and store the data,
at least one computing device configured to process and analyze the data stored in said one or more databases whereby the processing is based on the nature of the sequestration process and whereby an output of the processing is a determination of an amount of carbon dioxide removed from the air, and
a ledger database configured to store the output of the processing of said computing device, said ledger database thus configured to store the determination of the amount of carbon dioxide removed from the air;
an auditor facility having:
a data replication service configured to form a trust relationship and exchange cryptographic keys with said one or more databases of said sequestration facility to securely exchange the data stored by said one or more databases,
a continuous verification service configured to process and analyze the data stored by said data replication service whereby an output of the processing of said continuous verification service is a second determination of an amount of carbon dioxide removed from the air, and
an attestation service configured to form a trust relationship and exchange cryptographic keys to securely exchange the data stored by said ledger database, wherein said attestation service and said ledger database cooperate to define a first distributed ledger recording the amount of CO₂ removed from the air; and
a token generation and management facility having:
a token minting service configured to variably generate tokens based on the data contained in the ledger database and also based on a number of tokens previously generated, wherein said token minting service generates tokens such that an increasing volume of CO₂ corresponds to a minted token as more tokens are minted, and
a token management service configured to maintain data associated with at least one holder of each token generated by said token minting service.

4. The system of claim 3 further comprising:
at least one first sensor positioned and configured to monitor an identity of a gas passing through a pipeline out of said sequestration facility and further configured to confirm the gas is CO₂ and the volumetric flow rate of the gas;
at least one second sensor positioned and configured to monitor a concentration of CO₂ in a target formation to which sequestered CO₂ is directed, further configured to detect ruptures in rock that seals the target formation, and further configured to track migration of CO₂ from a position at which the CO₂ is injected into the target formation; and
at least one third sensor positioned and configured to monitor CO₂ levels at a ground surface above the target formation to detect leakage of CO₂.

5. A method of variably generating tokens in response to the sequestration of carbon dioxide comprising:
removing carbon dioxide from the air and sequestering the carbon dioxide in the ground;
recording data associated with said removing;
determining an amount of carbon dioxide sequestered based on the data and storing the amount in a first distributed ledger;
minting one or more tokens based on the amount in the first distributed ledger and based on a number of previously-minted tokens such that an increasing volume of CO 2 corresponds to a minted token as more tokens are minted; and
managing the one or more tokens with a second distributed ledger.

6. The method of claim 5 wherein said minting is further defined as:
minting one or more tokens based on the amount in the first distributed ledger and based on a number of previously-minted tokens such that an increasing volume of CO 2 corresponds to a minted token as more tokens are minted whereby a difficulty of minting each token increases at an exponential rate and not a hyperbolic rate.
